SEQUENCE LISTING (142 sequences)

SEQ ID NO: 1 - protein, 247 aa, synthetic construct
MLQNSAVLLVLVISASATHEAEQNDSVSPRKSRVAAQNSAEVVRCLNSALQVGCGAFACLENSTCDTDGMYDICKSFLYSAAKFDTQGKAFVKESLKCIANGVTSKVFLAIRRCSTFQRMIAEVQEECYSKLNVCSIAKRNPEAITEVVQLPNHFSNRYYNRLVRSLLECDEDTVSTIRDSLMEKIGPNMASLFHILQTDHCAQTHPRADFNRRRTNEPQKLKVLLRNLRGEEDSPSHIKRTSHESA

SEQ ID NO: 2 - DNA, 20 bp, synthetic construct
  source 1..20: mol_type = other DNA
cacgaggcggaacagaatga

SEQ ID NO: 3 - DNA, 20 bp, synthetic construct
  source 1..20: mol_type = other DNA
ccaacctgtagggcactgtt

SEQ ID NO: 4 - DNA, 20 bp, synthetic construct
  source 1..20: mol_type = other DNA
atggcgaccaccaaagtcaa

SEQ ID NO: 5 - DNA, 20 bp, synthetic construct
  source 1..20: mol_type = other DNA
gcagtgacgctcataaggga

SEQ ID NO: 6 - DNA, 20 bp, synthetic construct
  source 1..20: mol_type = other DNA
tgtcaatcctgtccgtgtcc

SEQ ID NO: 7 - DNA, 20 bp, synthetic construct
  source 1..20: mol_type = other DNA
acggaccagagcgaaagcat

SEQ ID NO: 8 - DNA, 21 bp, synthetic construct
  source 1..21: mol_type = other DNA
agagtgtgtctacttctgcca

SEQ ID NO: 9 - DNA, 22 bp, synthetic construct
  source 1..22: mol_type = other DNA
cttccaagtccatacggaacaa

SEQ ID NO: 10 - DNA, 18 bp, synthetic construct
  source 1..18: mol_type = other DNA
aatcaggtgacggtgttg

SEQ ID NO: 11 - DNA, 20 bp, synthetic construct
  source 1..20: mol_type = other DNA
acactccaggatcagtcttg

SEQ ID NO: 12 - DNA, 19 bp, synthetic construct
  source 1..19: mol_type = other DNA
cggtggctgtcagtcaaag

SEQ ID NO: 13 - DNA, 21 bp, synthetic construct
  source 1..21: mol_type = other DNA
aaacctcggcttcctccataa

SEQ ID NO: 14 - DNA, 20 bp, synthetic construct
  source 1..20: mol_type = other DNA
tggacaggatggattgccag

SEQ ID NO: 15 - DNA, 23 bp, synthetic construct
  source 1..23: mol_type = other DNA
ggggacctctttcacccttaaaa

SEQ ID NO: 16 - DNA, 20 bp, synthetic construct
  source 1..20: mol_type = other DNA
tgatggcgaagcgagtgaag

SEQ ID NO: 17 - DNA, 21 bp, synthetic construct
  source 1..21: mol_type = other DNA
actcatccatacacaggaccc

SEQ ID NO: 18 - DNA, 20 bp, synthetic construct
  source 1..20: mol_type = other DNA
cgagcggacctactgtccta

SEQ ID NO: 19 - DNA, 20 bp, synthetic construct
  source 1..20: mol_type = other DNA
gcccagtcaagtgtcaggtg

SEQ ID NO: 20 - DNA, 21 bp, synthetic construct
  source 1..21: mol_type = other DNA
gctggtctgatttctaccgca

SEQ ID NO: 21 - DNA, 21 bp, synthetic construct
  source 1..21: mol_type = other DNA
tccagcagagcacaagatgtc

SEQ ID NO: 22 - DNA, 21 bp, synthetic construct
  source 1..21: mol_type = other DNA
aagcagacagacgtgatctgg

SEQ ID NO: 23 - DNA, 20 bp, synthetic construct
  source 1..20: mol_type = other DNA
gcacgatgagccgaaagaag

SEQ ID NO: 24 - DNA, 20 bp, synthetic construct
  source 1..20: mol_type = other DNA
ggacttccttcgcaagagcc

SEQ ID NO: 25 - DNA, 20 bp, synthetic construct
  source 1..20: mol_type = other DNA
cgaagtgaaggagctgctgg

SEQ ID NO: 26 - DNA, 19 bp, synthetic construct
  source 1..19: mol_type = other DNA
gagcgcgtcgtaccgtatg

SEQ ID NO: 27 - DNA, 21 bp, synthetic construct
  source 1..21: mol_type = other DNA
actgacatctaactgcctggt

SEQ ID NO: 28 - DNA, 21 bp, synthetic construct
  source 1..21: mol_type = other DNA
atgtggacccctcctgatagt

SEQ ID NO: 29 - DNA, 21 bp, synthetic construct
  source 1..21: mol_type = other DNA
gcccagtgatttcagcaaagg

SEQ ID NO: 30 - DNA, 20 bp, synthetic construct
  source 1..20: mol_type = other DNA
atttccccggcaaagttcag

SEQ ID NO: 31 - DNA, 21 bp, synthetic construct
  source 1..21: mol_type = other DNA
aacggagtctcatgcagtaga

SEQ ID NO: 32 - DNA, 22 bp, synthetic construct
  source 1..22: mol_type = other DNA
gaaaaaggcgaacaaggtcttc

SEQ ID NO: 33 - DNA, 20 bp, synthetic construct
  source 1..20: mol_type = other DNA
caggaggccctctatcacca

SEQ ID NO: 34 - DNA, 19 bp, synthetic construct
  source 1..19: mol_type = other DNA
gcgctgttctcctaacccg

SEQ ID NO: 35 - DNA, 21 bp, synthetic construct
  source 1..21: mol_type = other DNA
gagtccagactgtgatctccg

SEQ ID NO: 36 - DNA, 20 bp, synthetic construct
  source 1..20: mol_type = other DNA
tcagccatcacagtgttccc

SEQ ID NO: 37 - DNA, 21 bp, synthetic construct
  source 1..21: mol_type = other DNA
atagcccgcatagcgtatcag

SEQ ID NO: 38 - DNA, 20 bp, synthetic construct
  source 1..20: mol_type = other DNA
ggacgtgtgacagctatggc

SEQ ID NO: 39 - DNA, 21 bp, synthetic construct
  source 1..21: mol_type = other DNA
tcccagtgaatggtgcataca

SEQ ID NO: 40 - DNA, 20 bp, synthetic construct
  source 1..20: mol_type = other DNA
aatgccgcgtatcgacttct

SEQ ID NO: 41 - DNA, 20 bp, synthetic construct
  source 1..20: mol_type = other DNA
gaccacaagctgacggctct

SEQ ID NO: 42 - DNA, 20 bp, synthetic construct
  source 1..20: mol_type = other DNA
gaggcgatccctgcaaacaa

SEQ ID NO: 43 - DNA, 20 bp, synthetic construct
  source 1..20: mol_type = other DNA
gtccaggccctgagttcttc

SEQ ID NO: 44 - DNA, 20 bp, synthetic construct
  source 1..20: mol_type = other DNA
atgctaacaacgtcctgtca

SEQ ID NO: 45 - DNA, 20 bp, synthetic construct
  source 1..20: mol_type = other DNA
agagtacttgcgttctggag

SEQ ID NO: 46 - DNA, 20 bp, synthetic construct
  source 1..20: mol_type = other DNA
cccaatgagtaggctggaga

SEQ ID NO: 47 - DNA, 20 bp, synthetic construct
  source 1..20: mol_type = other DNA
tctggacccattccttcttg

SEQ ID NO: 48 - DNA, 20 bp, synthetic construct
  source 1..20: mol_type = other DNA
gaagcctggtatgaggatct

SEQ ID NO: 49 - DNA, 20 bp, synthetic construct
  source 1..20: mol_type = other DNA
actgaccactctgtttccgt

SEQ ID NO: 50 - DNA, 20 bp, synthetic construct
  source 1..20: mol_type = other DNA
gaccattggattgggctcac

SEQ ID NO: 51 - DNA, 20 bp, synthetic construct
  source 1..20: mol_type = other DNA
ctcgctctccgttcctatgt

SEQ ID NO: 52 - DNA, 19 bp, synthetic construct
  source 1..19: mol_type = other DNA
gctcctcttaggggccact

SEQ ID NO: 53 - DNA, 19 bp, synthetic construct
  source 1..19: mol_type = other DNA
ccacgtctcaccattgggg

SEQ ID NO: 54 - DNA, 20 bp, synthetic construct
  source 1..20: mol_type = other DNA
caaccacggccttccctact

SEQ ID NO: 55 - DNA, 23 bp, synthetic construct
  source 1..23: mol_type = other DNA
ttctgcaagtgcatcatcgttgt

SEQ ID NO: 56 - DNA, 20 bp, synthetic construct
  source 1..20: mol_type = other DNA
cagtcagaccgatatgtgaa

SEQ ID NO: 57 - DNA, 20 bp, synthetic construct
  source 1..20: mol_type = other DNA
tagaggacacattgtgagca

SEQ ID NO: 58 - DNA, 19 bp, synthetic construct
  source 1..19: mol_type = other DNA
gctacgacgtgggctacag

SEQ ID NO: 59 - DNA, 23 bp, synthetic construct
  source 1..23: mol_type = other DNA
ccctcacactcagatcatcttct

SEQ ID NO: 60 - DNA, 20 bp, synthetic construct
  source 1..20: mol_type = other DNA
tgagtggctgtcttttgacg

SEQ ID NO: 61 - DNA, 20 bp, synthetic construct
  source 1..20: mol_type = other DNA
ggttcatgtcatggatggtg

SEQ ID NO: 62 - DNA, 20 bp, synthetic construct
  source 1..20: mol_type = other DNA
cttggttccctggcgtactc

SEQ ID NO: 63 - DNA, 21 bp, synthetic construct
  source 1..21: mol_type = other DNA
acctgatccgtccacaaacag

SEQ ID NO: 64 - DNA, 20 bp, synthetic construct
  source 1..20: mol_type = other DNA
aggacaacaacggaaagggc

SEQ ID NO: 65 - DNA, 20 bp, synthetic construct
  source 1..20: mol_type = other DNA
atccgactgatcggcactct

SEQ ID NO: 66 - DNA, 22 bp, synthetic construct
  source 1..22: mol_type = other DNA
ggatgtcacacaatgcaattcg

SEQ ID NO: 67 - DNA, 22 bp, synthetic construct
  source 1..22: mol_type = other DNA
tcacagaacggcttcctcaggt

SEQ ID NO: 68 - DNA, 20 bp, synthetic construct
  source 1..20: mol_type = other DNA
atccctggtttcattaacct

SEQ ID NO: 69 - DNA, 20 bp, synthetic construct
  source 1..20: mol_type = other DNA
gctccataaagtcaccaaag

SEQ ID NO: 70 - DNA, 22 bp, synthetic construct
  source 1..22: mol_type = other DNA
gcaactgttcctgaactcaact

SEQ ID NO: 71 - DNA, 21 bp, synthetic construct
  source 1..21: mol_type = other DNA
atcttttggggtccgtcaact

SEQ ID NO: 72 - DNA, 21 bp, synthetic construct
  source 1..21: mol_type = other DNA
gctcttactgactggcatgag

SEQ ID NO: 73 - DNA, 20 bp, synthetic construct
  source 1..20: mol_type = other DNA
cgcagctctaggagcatgtg

SEQ ID NO: 74 - DNA, 20 bp, synthetic construct
  source 1..20: mol_type = other DNA
tcttctgctgcgtgacaacc

SEQ ID NO: 75 - DNA, 21 bp, synthetic construct
  source 1..21: mol_type = other DNA
gagaaaccagcttggaaccag

SEQ ID NO: 76 - DNA, 19 bp, synthetic construct
  source 1..19: mol_type = other DNA
ctggcacaaaagggacgag

SEQ ID NO: 77 - DNA, 20 bp, synthetic construct
  source 1..20: mol_type = other DNA
acgtggccgagaatttcacc

SEQ ID NO: 78 - DNA, 19 bp, synthetic construct
  source 1..19: mol_type = other DNA
gaccgagtgcggttcaaag

SEQ ID NO: 79 - DNA, 19 bp, synthetic construct
  source 1..19: mol_type = other DNA
cgcagggcacatccaactt

SEQ ID NO: 80 - DNA, 21 bp, synthetic construct
  source 1..21: mol_type = other DNA
gtaacttcgtgcctagcaaca

SEQ ID NO: 81 - DNA, 23 bp, synthetic construct
  source 1..23: mol_type = other DNA
cctttgtcagaatactgagcagc

SEQ ID NO: 82 - DNA, 21 bp, synthetic construct
  source 1..21: mol_type = other DNA
tcggccaaggcaatttcagag

SEQ ID NO: 83 - DNA, 22 bp, synthetic construct
  source 1..22: mol_type = other DNA
cgagcatacttgaaccgattct

SEQ ID NO: 84 - DNA, 21 bp, synthetic construct
  source 1..21: mol_type = other DNA
atgtctggcttgcacctagta

SEQ ID NO: 85 - DNA, 23 bp, synthetic construct
  source 1..23: mol_type = other DNA
ccccaaagcgagtaacaaattct

SEQ ID NO: 86 - DNA, 23 bp, synthetic construct
  source 1..23: mol_type = other DNA
aaggacctgtctaggtttgatgc

SEQ ID NO: 87 - DNA, 21 bp, synthetic construct
  source 1..21: mol_type = other DNA
tggcttcataggtgacttcca

SEQ ID NO: 88 - DNA, 22 bp, synthetic construct
  source 1..22: mol_type = other DNA
gagtgctgacatctgacattga

SEQ ID NO: 89 - DNA, 21 bp, synthetic construct
  source 1..21: mol_type = other DNA
ttggcttccgaaacaccaaac

SEQ ID NO: 90 - DNA, 22 bp, synthetic construct
  source 1..22: mol_type = other DNA
ctgttccagccctataacttcg

SEQ ID NO: 91 - DNA, 22 bp, synthetic construct
  source 1..22: mol_type = other DNA
gaatgaggttgcccaatactcc

SEQ ID NO: 92 - DNA, 23 bp, synthetic construct
  source 1..23: mol_type = other DNA
aacgagcaaagtttgaactgagg

SEQ ID NO: 93 - DNA, 23 bp, synthetic construct
  source 1..23: mol_type = other DNA
tcgaagagcaccgaatatactga

SEQ ID NO: 94 - DNA, 22 bp, synthetic construct
  source 1..22: mol_type = other DNA
tctagctcctataccaccacca

SEQ ID NO: 95 - DNA, 22 bp, synthetic construct
  source 1..22: mol_type = other DNA
tcgtctccaacttatctcctcc

SEQ ID NO: 96 - DNA, 23 bp, synthetic construct
  source 1..23: mol_type = other DNA
aaagtgtgtcctacatggtaagc

SEQ ID NO: 97 - DNA, 22 bp, synthetic construct
  source 1..22: mol_type = other DNA
tgatccttcagggttgtcagtt

SEQ ID NO: 98 - DNA, 22 bp, synthetic construct
  source 1..22: mol_type = other DNA
acacttgctgtcctacagtttg

SEQ ID NO: 99 - DNA, 23 bp, synthetic construct
  source 1..23: mol_type = other DNA
gaggagccagaatcttgtaaaca

SEQ ID NO: 100 - DNA, 21 bp, synthetic construct
  source 1..21: mol_type = other DNA
tattgcggccaatgtctttgc

SEQ ID NO: 101 - DNA, 23 bp, synthetic construct
  source 1..23: mol_type = other DNA
cactggagtgatagactcaacca

SEQ ID NO: 102 - DNA, 20 bp, synthetic construct
  source 1..20: mol_type = other DNA
agcaggtgatcttcgaggag

SEQ ID NO: 103 - DNA, 20 bp, synthetic construct
  source 1..20: mol_type = other DNA
catggggcgaaaccaatgta

SEQ ID NO: 104 - DNA, 23 bp, synthetic construct
  source 1..23: mol_type = other DNA
ttatgggatcacagactaagcga

SEQ ID NO: 105 - DNA, 21 bp, synthetic construct
  source 1..21: mol_type = other DNA
tgctcctgtcatggttggaga

SEQ ID NO: 106 - DNA, 20 bp, synthetic construct
  source 1..20: mol_type = other DNA
cagcagctcctcgatgctac

SEQ ID NO: 107 - DNA, 21 bp, synthetic construct
  source 1..21: mol_type = other DNA
tcagacaggtcgggataaggg

SEQ ID NO: 108 - DNA, 19 bp, synthetic construct
  source 1..19: mol_type = other DNA
ggctgtgaccggaactgtg

SEQ ID NO: 109 - DNA, 22 bp, synthetic construct
  source 1..22: mol_type = other DNA
aggtctccaactggcattagaa

SEQ ID NO: 110 - DNA, 22 bp, synthetic construct
  source 1..22: mol_type = other DNA
aagacagtggttcagttggaag

SEQ ID NO: 111 - DNA, 22 bp, synthetic construct
  source 1..22: mol_type = other DNA
tgagttcggtcacagacttgat

SEQ ID NO: 112 - DNA, 21 bp, synthetic construct
  source 1..21: mol_type = other DNA
cttcgatggctatgtcagcga

SEQ ID NO: 113 - DNA, 21 bp, synthetic construct
  source 1..21: mol_type = other DNA
agcacgtcacctgagaggtag

SEQ ID NO: 114 - DNA, 22 bp, synthetic construct
  source 1..22: mol_type = other DNA
accaaagtgcaatcaaagtgga

SEQ ID NO: 115 - DNA, 21 bp, synthetic construct
  source 1..21: mol_type = other DNA
atgagggagttggaaggctct

SEQ ID NO: 116 - DNA, 21 bp, synthetic construct
  source 1..21: mol_type = other DNA
tctgcaacatggctagagact

SEQ ID NO: 117 - DNA, 20 bp, synthetic construct
  source 1..20: mol_type = other DNA
tccaagcattcgttggtccc

SEQ ID NO: 118 - DNA, 21 bp, synthetic construct
  source 1..21: mol_type = other DNA
tccagttggcttatcgtggtg

SEQ ID NO: 119 - DNA, 23 bp, synthetic construct
  source 1..23: mol_type = other DNA
tccagagtccgattgatttttgc

SEQ ID NO: 120 - DNA, 22 bp, synthetic construct
  source 1..22: mol_type = other DNA
catgtatcgccgtaaactggac

SEQ ID NO: 121 - DNA, 21 bp, synthetic construct
  source 1..21: mol_type = other DNA
tggtaggagcacataggcact

SEQ ID NO: 122 - DNA, 22 bp, synthetic construct
  source 1..22: mol_type = other DNA
tggataaccaacggaggaaaag

SEQ ID NO: 123 - DNA, 20 bp, synthetic construct
  source 1..20: mol_type = other DNA
ctggggtatctgcttccaca

SEQ ID NO: 124 - DNA, 19 bp, synthetic construct
  source 1..19: mol_type = other DNA
actcgcagccagcgttatg

SEQ ID NO: 125 - DNA, 19 bp, synthetic construct
  source 1..19: mol_type = other DNA
agggtcagcgatgccaaac

SEQ ID NO: 126 - DNA, 20 bp, synthetic construct
  source 1..20: mol_type = other DNA
ctcatagctggacggctgtt

SEQ ID NO: 127 - DNA, 20 bp, synthetic construct
  source 1..20: mol_type = other DNA
ggcccaaggtagctggaaat

SEQ ID NO: 128 - DNA, 20 bp, synthetic construct
  source 1..20: mol_type = other DNA
ttcagcaactgggtctgcaa

SEQ ID NO: 129 - DNA, 20 bp, synthetic construct
  source 1..20: mol_type = other DNA
aagaacacgtaaggcccgag

SEQ ID NO: 130 - DNA, 20 bp, synthetic construct
  source 1..20: mol_type = other DNA
gtggtagagctttggggacg

SEQ ID NO: 131 - DNA, 20 bp, synthetic construct
  source 1..20: mol_type = other DNA
actatgcaagctgtgaggca

SEQ ID NO: 132 - DNA, 20 bp, synthetic construct
  source 1..20: mol_type = other DNA
agtgggctgtaccaacatcg

SEQ ID NO: 133 - DNA, 20 bp, synthetic construct
  source 1..20: mol_type = other DNA
caggcctcgaagtccgttag

SEQ ID NO: 134 - DNA, 25 bp, synthetic construct
  source 1..25: mol_type = other DNA
cccagactagagatcctgacagaat

SEQ ID NO: 135 - DNA, 23 bp, synthetic construct
  source 1..23: mol_type = other DNA
gcacaacgctcttttcttttacc

SEQ ID NO: 136 - DNA, 20 bp, synthetic construct
  source 1..20: mol_type = other DNA
gcctgtgggaaaaccaacct

SEQ ID NO: 137 - DNA, 22 bp, synthetic construct
  source 1..22: mol_type = other DNA
cacccacacattcaactttcca

SEQ ID NO: 138 - DNA, 20 bp, synthetic construct
  source 1..20: mol_type = other DNA
agggacggatgtgtgtcaac

SEQ ID NO: 139 - DNA, 20 bp, synthetic construct
  source 1..20: mol_type = other DNA
catcaaacacggtccgcaag

SEQ ID NO: 140 - DNA, 23 bp, synthetic construct
  source 1..23: mol_type = other DNA
ggagagtgcagatacctgaagga

SEQ ID NO: 141 - DNA, 21 bp, synthetic construct
  source 1..21: mol_type = other DNA
gtcagcccatgacacctctgt

SEQ ID NO: 142 - DNA, 2481 bp, synthetic construct
  source 1..2481: mol_type = other DNA
caagaagcagcgactgcagcagcagcagcagcagcggcggtggcagcagcagcagcagcggcggcagcagcagcagcagcggaggcaccggtggcagcagcagcatcaccagcaacaacaacaaaaaaaaatcctcatcaaatcctcacctaagctttcagtgtatccagatccacatcttcactcaagccaggagagggaaagaggaaaggggggcaggaaaaaaaaaaaacccaacaacttagcggaaacttctcagagaatgctccaaaactcagcagtgcttctggtgctggtgatcagtgcttctgcaacccatgaggcggagcagaatgactctgtgagccccaggaaatcccgagtggcggctcaaaactcagctgaagtggttcgttgcctcaacagtgctctacaggtcggctgcggggcttttgcatgcctggaaaactccacctgtgacacagatgggatgtatgacatctgtaaatccttcttgtacagcgctgctaaatttgacactcagggaaaagcattcgtcaaagagagcttaaaatgcatcgccaacggggtcacctccaaggtcttcctcgccattcggaggtgctccactttccaaaggatgattgctgaggtgcaggaagagtgctacagcaagctgaatgtgtgcagcatcgccaagcggaaccctgaagccatcactgaggtcgtccagctgcccaatcacttctccaacagatactataacagacttgtccgaagcctgctggaatgtgatgaagacacagtcagcacaatcagagacagcctgatggagaaaattgggcctaacatggccagcctcttccacatcctgcagacagaccactgtgcccaaacacacccacgagctgacttcaacaggagacgcaccaatgagccgcagaagctgaaagtcctcctcaggaacctccgaggtgaggaggactctccctcccacatcaaacgcacatcccatgagagtgcataaccagggagaggttattcacaacctcaccaaactagtatcattttaggggtgttgacacaccagttttgagtgtactgtgcctggtttgatttttttaaagtagttcctattttctatcccccttaaagaaaattgcatgaaactaggcttctgtaatcaatatcccaacattctgcaatggcagcattcccaccaacaaaatccatgtgaccattctgcctctcctcaggagaaagtaccctcttttaccaacttcctctgccatgtttttcccctgctcccctgagaccacccccaaacacaaaacattcatgtaactctccagccattgtaatttgaagatgtggatccctttagaacggttgccccagtagagttagctgataaggaaactttatttaaatgcatgtcttaaatgctcataaagatgttaaatggaattcgtgttatgaatctgtgctggccatggacgaatatgaatgtcacatttgaattcttgatctctaatgagctagtgtcttatggtcttgatcctccaatgtctaattttctttccgacacatttaccaaattgcttgagcctggctgtccaaccagactttgagcctgcatcttcttgcatctaatgaaaaacaaaaagctaacatctttacgtactgtaactgctcagagctttaaaagtatctttaacaattgtcttaaaaccagagaatcttaaggtctaactgtggaatataaatagctgaaaactaatgtactgtacataaattccagaggactctgcttaaacaaagcagtatataataactttattgcatatagatttagttttgtaacttagctttatttttcttttcctgggaatggaataactatctcacttccagatatccacataaatgctccttgtggccttttttataactaagggggtagaagtagttttaattcaacatcaaaacttaagatgggcctgtatgagacaggaaaaaccaacaggtttatctgaaggaccccaggtaagatgttaatctcccagcccacctcaacccagaggctactcttgacttagacctatactgaaagatctctgtcacatccaactggaaattccaggaaccaaaaagagcatccctatgggcttggaccacttacagtgtgataaggcctactatacattaggaagtggcagttctttactcgtcccctttcatcggtgcctggtactctggcaaatgatgatggggtgggagactttccattaaatcaatcaggaatgagtcaatcagcctttaggtctttagtccgggggacttggggctgagagagtataaataaccctgggctgtccagccttaatagacttctcttacattttcgtcctgtagcacgctgcctgccaaagtagtcctggcagctggaccatctctgtaggatcgtaaaaaaatagaaaaaaaaaaaaaaaaaaaaaaaaa

(19) United States
(12) Patent Application Publication
(10) Pub. No.: US 2024/0325497 A1
(43) Pub. Date: Oct. 3, 2024

(54) COMPOSITION FOR PREVENTING OR TREATING LIVER DISEASES, COMPRISING STC-1 OR DERIVATIVE THEREOF

(71) Applicant: INDUSTRY-ACADEMIC COOPERATION FOUNDATION, YONSEI UNIVERSITY, Seoul (KR)

(72) Inventors: Cheol Ryong KU, Seoul (KR); Ju Hun OH, Seoul (KR); Chan Woo KANG, Gyeonggi-do (KR); Eun Kyung WANG, Seoul (KR); Zhenyu HONG, Seoul (KR); Yong Ho LEE, Seoul (KR)

(21) Appl. No.: 18/580,971

(22) PCT Filed: Aug. 3, 2022
(86) PCT No.: PCT/KR2022/011447

§ 371 Date: Jan. 19, 2024

(30) Foreign Application Priority Data
Aug. 3, 2021 (KR) 10-2021-0101932

(51) Int. Cl. A61K 38/22; A61K 9/00; A61P 1/16

(52) U.S. Cl. CPC A61K 38/22; A61K 9/0056; A61P 1/16

(57) ABSTRACT

The present invention relates to a pharmaceutical composition for preventing or treating liver disease comprising STC-1 or a derivative thereof as an active ingredient. The STC-1 protein according to the present invention reduces the expression of liver fibrosis markers ALT and Lect2 and reduces the area of fibrotic tissue, in animal models of liver fibrosis, indicating that it has excellent effects on the prevention and treatment of liver fibrosis and, furthermore, cirrhosis.

### TECHNICAL FIELD

The present invention relates to a composition for preventing or treating liver disease, and more specifically, to a pharmaceutical composition for preventing or treating liver disease comprising STC-1 or a derivative thereof as an active ingredient.

### BACKGROUND ART

Liver fibrosis can be defined as excessive accumulation of extracellular matrix proteins due to chronic inflammation of the liver. If this chronic inflammation of the liver persists, liver fibrosis develops into cirrhosis due to a decrease in the number of liver cells.

Representative cells involved in liver fibrosis include hepatic stellate cells, Kupffer cells, and endothelial cells. Hepatic stellate cells are the main source of extracellular matrix production and are involved in increasing the production of various extracellular matrix proteins, including collagen. Kupffer cells are located in the sinusoidal space in the liver, and substances produced by activated Kupffer cells affect surrounding hepatic cells, endothelial cells, and hepatic stellate cells, thereby promoting liver fibrosis.

Endothelial cells not only play an essential role in the regulation of blood flow in the liver, but also are involved in the production of growth factors and extracellular matrix involved in the proliferation of hepatic stellate cells by inflammation or liver fibrosis.

The representative intracellular signaling involved in liver fibrosis occurs through TGF-β, the most powerful fibrosis-promoting cytokine in hepatic stellate cells. TGF-β produced from hepatic stellate cells binds to type II receptors, which in turn phosphorylate type I receptors, which then recruit and phosphorylate smad2 and smad3. Then, the smad2/smad3 complex binds with smad4 and then translocates into the nucleus to regulate the transcription of target genes. On the other hand, Smad7 interacts with type I receptors to interfere with phosphorylation of smad2 and smad3 and interferes with TGF-β-induced smad signaling, thereby delaying liver fibrosis.

Until now, liver fibrosis has been known to be an irreversible process, but recent clinical and experimental studies have reported that liver fibrosis is a reversible process, suggesting that the degree of liver fibrosis can be controlled. Therefore, research on inhibiting the progression of liver fibrosis through activated hepatic stellate cells began to receive attention.

Meanwhile, evolutionarily conserved mammalian stanniocalcin-1 (STC-1) is a secreted glycoprotein that is expressed in various tissues, including human ovaries, kidneys, brain, lungs, heart, muscles, and bones. STC-1, which has a hormone-like function, is known to be secreted extracellularly or act intracellularly and involved in calcium homeostasis and oxidative stress control responses. It is known that extracellular STC-1 is secreted into the blood, enters cells through endocytosis, and mostly binds to the inner mitochondrial membrane, thereby regulating superoxide production in mitochondria (Prior Art Documents 1, 2 and 3).

Studies have recently been conducted on mammalian STC-1, in addition to the well-known role of STC-1 in regulating blood calcium levels. Recently, various roles have been reported, including wound healing, cell muscle and bone development, metabolism, angiogenesis, cancer metabolism, and fibrosis (Prior Art Documents 2 to 17). It is known that, through functions represented by antioxidant and anti-inflammatory responses, STC-1 reduces the infiltration and differentiation of immune cells, exhibits therapeutic effects on cardiac and renal kidney inflammatory responses, and alleviates pulmonary fibrosis (Prior Art Documents 2 to 17). However, although STC-1 has received attention as a treatment factor for various diseases, studies on the effects thereof on the liver are still insufficient.

Accordingly, the present inventors have performed gastrectomy on diabetic animal models, and as a result, have found that the expression of STC-1 increased in experimental animals with induced steatohepatitis. In addition, the present inventors have found that, when STC-1 protein was administered to mouse models of liver fibrosis, the expression of fibrosis- and inflammation-related genes decreased and fibrotic lesions decreased, thereby completing the present invention.

### PRIOR ART DOCUMENTS

### Non-Patent Documents

(Non-Patent Document 1) 1. Ellard J P et al., Mol Cell Endocrinol. 264:90-101, 2007
(Non-Patent Document 2) 2. Wang Y et al., J Leukoc Biol., 86:981-8, 2009
(Non-Patent Document 3) 3. Sheikh-Hamad D et al., Am J Physiol Renal Physiol., 298 (2): F248-54, 2010
(Non-Patent Document 4) 4. Yeung B H et al., PLOS One, 6 (11): e27094, 2011.
(Non-Patent Document 5) 5. Sarapio E et al., Molecular and Cellular Endocrinology, 485:81-87, 2019
(Non-Patent Document 6) 6. Iyer V R et al., Science, 283 (5398): 83-7, 1999
(Non-Patent Document 7) 7. McCudden C R et al., J Biol Chem., 277 (47): 45249-58, 2002.
(Non-Patent Document 8) 8. Kahn J et al., Am J Pathol., 156 (6): 1887-900, 2000
(Non-Patent Document 9) 9. Filvaroff E H et al., Endocrinology, 156 (6): 1887-900, 2002
(Non-Patent Document 10) 10. Serlachius M et al., Br J Haematol, 119 (2): 359-63, 2002
(Non-Patent Document 11) 11. Chang A C et al., Endocr Relat Cancer, 10 (3): 359-73, 2003
(Non-Patent Document 12) 12. Leung C C et al., Oncotarget, 9 (6): 6852-6861, 2017
(Non-Patent Document 13) 13. Mohammadipoor A et al., Transl Res., 177:127-142, 2016
(Non-Patent Document 14) 14. Lin H et al., Cancer Cell, 39 (4): 480-493.e6, 2021
(Non-Patent Document 15) 15. Kamata T et al., Cell Rep., 31 (12): 107802, 2020
(Non-Patent Document 16) 16. Huang L et al., Sci Rep., 5:18117, 2015
(Non-Patent Document 17) 17. Pan J S et al., J Am Soc Nephrol., 26 (2): 364-78, 2015
(Non-Patent Document 18) 18. Xu M et al., Cell, 78 (6): 1478-1492.e20, 2019

### SUMMARY OF THE INVENTION

An object of the present invention is to provide a use for preventing or treating liver disease.

Another object of the present invention is to provide a health functional food for preventing or ameliorating liver disease.

Still another purpose of the present invention is to provide health functional food for liver function improvement.

To achieve the above objects, the present invention provides a pharmaceutical composition for preventing or treating liver disease comprising STC-1 or a derivative thereof as an active ingredient.

The present invention also provides a health functional food for preventing or ameliorating liver disease comprising STC-1 or a derivative thereof as an active ingredient.

The present invention also provides a health functional food for improving liver function comprising STC-1 or a derivative thereof as an active ingredient.

The present invention also provides a method for preventing or treating liver disease comprising a step of administering STC-1 or a derivative thereof.

The present invention also provides the use of STC-1 or a derivative thereof for the prevention or treatment of liver disease.

The present invention also provides the use of amorphous STC-1 or a derivative for producing a medicament for preventing or treating liver disease.

### BRIEF DESCRIPTION OF DRAWINGS

FIG. 1 shows top 20 genes with a large difference in expression level, selected from among transcripts by sequencing of RNA from alimentary limb tissues 12 weeks after performing RYGB surgery after feeding an OLETF model with a high-fat diet.

FIG. 2 shows the results of measuring the expression level of STC-1 by ELISA analysis using, as a sample, plasma obtained from blood collected from the inferior vena cava and portal vein of each of a RYGB surgery group and a sham operation (SHAM) group.

FIG. 3 shows the results of measuring, by qRT-PCR, the difference in STC-1 expression between a segment with increased FDG uptake and a segment without increased FDG uptake in alimentary limb tissues, 12 weeks after performing RYGB surgery after feeding an OLETF model with a high-fat diet.

FIG. 4 shows the results of measuring, by qRT-PCR, changes in expression of fat metabolism-related genes in liver cancer HepG cells in a group treated with free fatty acid (FFA) and a group treated with free fatty acid plus STC-1.

FIG. 5 shows the results of measuring, by qRT-PCR, changes in expression of inflammation- and fibrosis-related genes in liver cancer HepG cells in a group treated with free fatty acid (FFA) and a group treated with free fatty acid plus STC-1.

FIG. 6 shows the results of measuring, by qRT-PCR, changes in expression of inflammation- and fibrosis-related markers, after treating hepatic stellate LX-2 cells (that causes liver fibrosis) with TGF-beta to induce fibrosis and activation and treating the cells with STC-1 and GDF-15.

FIG. 7 shows the results of measuring, by Western blot analysis, the amounts of t-AMPKα, p-AMPKα and STC-1 proteins in cells obtained by overexpressing STC-1 in HepG2 cells, followed by treatment with palmitic acid.

FIG. 8 shows the results of measuring, by qRT-PCR, changes in expression of glucose metabolism-related genes, after overexpressing (O/E) STC-1 in intestinal epithelial IEC18 cells or adding STC-1 to the cells and culturing the cells.

FIG. 9 shows the results of measuring serum AST and ALT levels by ELISA (top) and the results of measuring the expression of Lect2 in liver tissue by qPCR (bottom), in mice generated by administering STC-1 to mouse models of liver fibrosis induced by MCD diet feeding.

FIG. 10 shows the results of measuring, by qPCR, the expression of liver fibrosis- and inflammation-related genes in mice generated by administering STC-1 to mouse models of liver fibrosis induced by MCD diet feeding.

FIG. 11 shows the results of Sirius red staining of the livers harvested from mice generated by administering STC-1 to mouse models of liver fibrosis induced by MCD diet feeding.

FIG. 12 shows the results of measuring, by qRT-PCR, the expression of inflammatory cytokines in STC-1-overexpressing HepG2 cells (upper well) and LPS-treated macrophage Raw264.7 cells (lower well) in a Transwell insert.

FIG. 13 shows the results of measuring, by qRT-PCR, the changes in expression of inflammatory cytokines by hSTC-1 treatment in macrophages with LPS-induced inflammation.

FIG. 14 shows the results of measuring, by qRT-PCR, changes in expression of inflammatory cytokines after treating inflammation-induced macrophages with different concentrations of hSTC-1.

### DETAILED DESCRIPTION AND PREFERRED EMBODIMENTS OF THE INVENTION

Unless otherwise defined, all technical and scientific terms used in the present specification have the same meanings as commonly understood by those skilled in the art to which the present disclosure pertains. In general, the nomenclature used in the present specification is well known and commonly used in the art.

In the present invention, gastrectomy was performed on diabetic animal models, and as a result, it was found that the expression of STC-1 increased in experimental animals with induced steatohepatitis, suggesting that STC-1 would play an important role in the progression of liver disease. Moreover, was it found that LPS-induced inflammatory responses of macrophages were inhibited by STC-1-overexpressing liver cells. In addition, it was found that, when STC-1 protein was administered to a mouse model of liver fibrosis, the expression of fibrosis- and inflammation-related genes decreased and fibrotic lesions decreased.

Therefore, in one aspect, the present invention is directed to a pharmaceutical composition for preventing or treating liver disease comprising STC-1 or a derivative thereof as an active ingredient.

In the present invention, the liver disease may be liver fibrosis, cirrhosis, acute hepatitis, chronic hepatitis, fatty liver, or liver cancer, without being limited thereto.

In the present invention, the STC-1 protein may be characterized by having an amino acid sequence represented by SEQ ID NO: 1, without being limited thereto, and any protein having the activity of STC-1 while having sequence similarity to SEQ ID NO: 1 may be used without limitation.

SEQ ID NO. 1:
MLQNSAVLLVLVISASATHEAEQNDSVSPRKSRVAAQNSAEVVRCLNSA
LQVGCGAFACLENSTCDTDGMYDICKSFLYSAAKFDTQGKAFVKESLKC
IANGVTSKVFLAIRRCSTFQRMIAEVQEECYSKLNVCSIAKRNPEAITE
VVQLPNHFSNRYYNRLVRSLLECDEDTVSTIRDSLMEKIGPNMASLFHI
LQTDHCAQTHPRADFNRRRTNEPQKLKVLLRNLRGEEDSPSHIKRTSHE
SA

In the present invention, the STC-1 protein may be characterized by having at least 70% homology, at least 80% homology, at least 90% homology, at least 95% homology, at least 96% homology, at least 97% homology, at least 98% homology, or at least 99% homology to SEQ ID NO: 1.

The STC-1 protein or derivative thereof in the present invention is a concept covering a fragment of the STC-1 protein in which one or more amino acids at the N-terminus or C-terminus in the amino acid sequence of STC-1 of SEQ ID NO: 1 are truncated and which still retains the activity of the STC-1 protein. Furthermore, the STC-1 protein or derivative thereof is a concept covering an STC-1 protein which includes a substitution (preferably conservative substitution) of one or more amino acids in the STC-1 protein and still retains the activity of the STC-1 protein.

As used herein, “conservative substitutions” refers to modifications of a polypeptide that involve the substitution of one or more amino acids for amino acids having similar biochemical properties that do not result in loss of a biological or biochemical function of the polypeptide. A “conservative amino acid substitution” is one in which the amino acid residue is replaced with an amino acid residue having a similar side chain. Families of amino acid residues having similar side chains have been defined in the art and are well known. These families include amino acids with basic side chains (e.g., lysine, arginine, histidine), amino acids with acidic side chains (e.g., aspartic acid, glutamic acid), amino acids with uncharged polar side chains (e.g., glycine, asparagine, glutamine, serine, threonine, tyrosine, cysteine), amino acids with nonpolar side chains (e.g., alanine, valine, leucine, isoleucine, proline, phenylalanine, methionine, tryptophan), amino acids with ß-branched side chains (e.g., threonine, valine, isoleucine), and amino acids with aromatic side chains (e.g., tyrosine, phenylalanine, tryptophan, histidine). The STC-1 protein of the present invention is expected to have conservative amino acid substitutions and still retain activity, and proteins in which conservative substitution mutations occurred are also included in the scope of the present invention, as long as they retain the characteristics of the STC-1 protein according to the present invention.

The composition of the present invention may further contain pharmaceutically acceptable additives. In this case, examples of pharmaceutically acceptable additives that may be used include starch, gelatinized starch, microcrystalline cellulose, lactose, povidone, colloidal silicon dioxide, calcium hydrogen phosphate, lactose, mannitol, taffy, gum arabic, pregelatinized starch, corn starch, powdered cellulose, hydroxypropyl cellulose, Opadry, sodium starch glycolate, carnauba wax, synthetic aluminum silicate, stearic acid, magnesium stearate, aluminum stearate, calcium stearate, white sugar, and the like. The pharmaceutically acceptable additives according to the present invention are preferably contained in an amount of 0.1 to 90 parts by weight based on the total weight of the composition, without being limited thereto.

The composition of the present invention may be administered in various oral or parenteral dosage forms at the time of actual clinical administration. For formulation, the composition may be prepared using commonly used diluents or excipients, such as fillers, extenders, binders, wetting agents, disintegrants, surfactants, etc. As suitable formulations known in the art, it is preferable to use those disclosed in Remington's Pharmaceutical Science, the latest edition, Mack Publishing Company, Easton PA. Carriers, excipients and diluents, which may be contained in the composition, include lactose, dextrose, sucrose, oligosaccharides, sorbitol, mannitol, xylitol, erythritol, maltitol, starch, gum acacia, alginate, gelatin, calcium phosphate, calcium silicate, cellulose, methyl cellulose, microcrystalline cellulose, polyvinyl pyrrolidone, water, methyl hydroxybenzoate, propyl hydroxybenzoate, talc, magnesium stearate, mineral oil, etc.

Solid formulations for oral administration include tablets, pills, powders, granules, capsules, etc., and these solid formulations are prepared by mixing one or more excipients, such as starch, calcium carbonate, sucrose, lactose, gelatin, etc. In addition to simple excipients, lubricants such as magnesium stearate or talc are also used. Liquid formulations for oral administration include suspensions, solutions, emulsions, syrups, etc., and may contain various excipients, for example, wetting agents, flavoring agents, and preservatives, in addition to water and liquid paraffin, which are frequently used simple diluents.

Formulations for parenteral administration include sterile aqueous solutions, non-aqueous solutions, suspensions, emulsions, freeze-dried preparations, and suppositories.

As non-aqueous solvents or suspending agents, propylene glycol, polyethylene glycol, plant oils such as olive oil, injectable esters such as ethyl oleate, and the like may be used. As bases for suppositories, Witepsol, Macrogol, Tween 61, cacao butter, laurin fat, glycerogelatin, and the like may be used. Parenteral administration may be performed using an external injection, intraperitoneal injection, intrarectal injection, subcutaneous injection, intravenous injection, intramuscular injection, or intrathoracic injection method.

The dosage of the pharmaceutical composition of the present invention varies depending on the patient's weight, age, gender, health condition, diet, time of administration, mode of administration, rate of excretion, and severity of the disease, and may be administered once or several times a day.

The pharmaceutical composition of the present invention may be administered to a subject through various routes. All modes of administration can be contemplated, and for example, the pharmaceutical composition may be administered orally, intrarectally, or by intravenous, intramuscular, subcutaneous, intrauterine, intrathecal or intracerebroventricular injection.

The pharmaceutical compound of the present invention may be used alone or in combination with surgery, radiotherapy, hormone therapy, chemotherapy, and methods that use biological response modifiers.

In the present invention, the term “prevention” refers to any action that inhibits or delays liver disease by administering the pharmaceutical composition of the present invention, and the term “treatment” refers to any action that alleviates or beneficially alters symptoms of liver disease by the pharmaceutical composition of the present invention.

In one embodiment of the present invention, “prevention” may include, without limitation, any action capable of blocking, suppressing or delaying symptoms caused by liver disease by using the composition of the present invention.

In one embodiment of the present invention, “amelioration” may include, without limitation, any action that can alleviate or beneficially alter symptoms of liver disease by using the composition of the present invention.

In one embodiment of the present invention, “treatment” refers to a series of actions that are performed to alleviate or/and ameliorate the disease in question.

In addition, the pharmaceutical composition according to the present invention comprises the active ingredient of the present invention in an amount of 0.1 to 50 wt % based on the total weight of the composition. Carriers, excipients and diluents that may be contained in the composition of the present invention include, but are not limited to, lactose, dextrose, sucrose, oligosaccharides, sorbitol, mannitol, xylitol, erythritol, maltitol, starch, gum acacia, alginate, gelatin, calcium phosphate, calcium silicate, cellulose, methyl cellulose, microcrystalline cellulose, polyvinyl pyrrolidone, water, methyl hydroxybenzoate, propyl hydroxybenzoate, talc, magnesium stearate, and mineral oil.

In one embodiment of the present invention, “administration” means introducing the composition of the present invention to a patient by any suitable method. The composition of the present invention may be administered via any general route, as long as it can reach a target tissue.

The pharmaceutical composition of the present invention may be administered orally, intraperitoneally, intravenously, intramuscularly, subcutaneously, intradermally, intranasally, intrapulmonarily, enterically, intracavitarily, intraperitoneally, or intrathecally, without being limited thereto. In the present invention, the effective amount may be determined depending on various factors, including the type of disease, the severity of the disease, the types and contents of the active ingredient and other ingredients contained in the composition, the type of formulation, the patient's age, body weight, general health condition, sex, and diet, administration time, the route of administration, the secretion rate of the composition, the treatment period, and concurrently used drugs. In addition, the pharmaceutical composition of the present invention may be administered alone or together with other therapies known in the art, such as chemotherapy and surgery, for the treatment of a target liver disease. In addition, the pharmaceutical composition of the present invention may be administered in combination with other treatments designed to promote blood glucose lowering, for example, those well known in the art. Other standard delivery methods may also be used, such as biolistic delivery or ex vivo treatment.

In another aspect, the present invention is directed to a method for preventing or treating liver disease comprising a step of administering STC-1 or a derivative thereof.

In still another aspect, the present invention is directed to the use of STC-1 or a derivative thereof for the prevention or treatment of liver disease.

In yet another aspect, the present invention is directed to the use of STC-1 or a derivative thereof for producing a medicament for the prevention or treatment of liver disease.

In yet another aspect, the present invention is directed to the use of STC-1 or a derivative thereof for producing a medicament for the prevention or treatment of liver disease.

In still yet another aspect, the present invention is directed to a health functional food for preventing or ameliorating liver disease comprising STC-1 or a derivative thereof as an active ingredient.

In a further aspect, the present invention is directed to a health functional food for improving liver function comprising STC-1 or a derivative thereof as an active ingredient.

In the present invention, “health functional food” refers to food having biological modulation functions, such as prevention and alleviation of diseases, bio-defense, immunity, recovery after diseases, suppression of aging, or the like. The health functional food needs to be harmless to the human body when taken for a long period of time.

When the STC-1 protein of the present invention is used as a food additive, the STC-1 or derivative thereof may be added per se, or may be used together with another food or food ingredient, and may be properly used according to a conventional method. The amount of active ingredient added may be properly determined according to the purpose of use (prevention, health, or therapeutic treatment). In general, the STC-1 or derivative thereof according to the present invention is generally added in an amount of 15 wt % or less, preferably 10 wt % or less, relative to the raw materials when a food or beverage is prepared. However, when the active ingredient is taken for a long period of time for the purpose of health and sanitation or health control, the amount of the active ingredient may be below the lower limit of the above range. Since the active ingredient is not problematic in terms of safety, the active ingredient may be used in an amount above the upper limit of the above range.

When the STC-1 or derivative thereof according to the present invention is used as a food additive, the STC-1 protein or derivative thereof may be added per se, or may be used together with another food or food ingredient, and may be properly used according to a conventional method. The amount of active ingredient added may be properly determined according to the purpose of use (prevention, health, or therapeutic treatment). In general, the STC-1 protein or derivative thereof according to the present invention is generally added in an amount of 15 wt % or less, preferably 10 wt % or less, relative to the raw materials when a food or beverage is prepared. However, when the active ingredient is taken for a long period of time for the purpose of health and sanitation or health control, the amount of the active ingredient may be below the lower limit of the above range. Since the active ingredient is not problematic in terms of safety, the active ingredient may be used in an amount above the upper limit of the above range.

The health beverage composition may contain various flavoring agents or natural carbohydrates as additional ingredients, like conventional beverages. Examples of the natural carbohydrates include monosaccharides (e.g., glucose, fructose, etc.), disaccharides (e.g., maltose, sucrose, etc.), dextrin, cyclodextrin, etc., and examples of the flavoring agents include synthetic flavoring agents such as saccharin and aspartame. The proportion of the natural carbohydrates is generally about 0.01 to 10 g, preferably about 0.01 to 0.1 g, per 100 ml of the composition of the present invention, without being limited thereto.

In addition, the composition of the present invention may contain various nutrients, vitamins, electrolytes, flavoring agents, colorants, pectic acid and salts thereof, alginic acid and salts thereof, organic acids, protective colloidal thickeners, pH adjusting agents, stabilizers, preservatives, glycerin, alcohols, carbonating agents used in carbonated beverages, and the like. In addition, the composition of the present invention may contain fruit flesh for manufacturing natural fruit juice, fruit juice beverages, and vegetable beverages. These ingredients may be used independently or in combination. The proportion of these additives is not greatly critical, but is generally selected within the range of 0.01 to 0.1 parts by weight based on 100 parts by weight of the composition of the present invention.

In one embodiment of the present invention, the “food composition” is variously used for the prevention or amelioration of a target liver disease in the present invention. The food composition comprising the composition of the present invention as an active ingredient may be prepared in the form of various foods, such as beverages, gum, tea, vitamin complexes, powders, granules, tablets, capsules, confectionery, rice cakes, bread, etc. The food composition of the present invention may be safely used even when taken for a long period of time for preventive purposes because it is an improved food composed of existing food components with little toxicity and side effects. When the composition of the present invention is contained in a food composition, it may be added in an amount of 0.1 to 100 wt % based on the total weight. Here, when the food composition is prepared in the form of a beverage, there is no particular limitation except that the food composition is contained at the indicated ratio. In this case, the food composition may contain various flavoring agents or natural carbohydrates as additional ingredients, like conventional beverages. Examples of the natural carbohydrates include conventional sugars, such as monosaccharides (e.g., glucose, etc.), disaccharides (e.g., sucrose, etc.), polysaccharides (e.g., dextrin, cyclodextrin, etc.), and sugar alcohols such as xylitol, sorbitol, erythritol or the like. Examples of the flavoring agents include natural flavoring agents (thaumatin, stevia extract (e.g., rebaudioside A, glycyrrhizin, etc.) and synthetic flavoring agents (saccharin, aspartame, etc.). In addition, the food composition of the present invention may contain various nutrients, vitamins, minerals (electrolytes), flavoring agents such as synthetic flavoring agents and natural flavoring agents, colorants, pectic acid and salts thereof, alginic acid and salts thereof, organic acids, protective colloidal thickeners, pH adjusting agents, stabilizers, preservatives, glycerin, alcohols, carbonizing agents that are used in carbonated beverages, etc. These components may be used independently or in combination. The proportion of these additives is generally selected within the range of 0.1 to 100 parts by weight based on 100 parts by weight of the composition of the present invention, without being limited thereto.

### EXAMPLES

Hereinafter, the present invention will be described in more detail with reference to examples. These examples are only for illustrating the present invention, and it will be apparent to those skilled in the art that the scope of the present invention is not to be construed as being limited by these examples.

### Example 1: Evaluation of Effect of Alleviating Steatohepatitis After Gastrectomy in Type 2 Diabetes Rat Animal Model OLETF (Otsuka Long-Evans Tokushima Fatty) Rat

An OLETF (Otsuka Long-Evans Tokushima Fatty) rat (Japan SLC, Inc.), a representative rat model of obesity and diabetes, is a rat model lacking the CCK-a gene and is characterized by a deficiency of CCK hormone, known as an appetite regulating hormone.

A total of 20 OLETF rats were fed a diet with a fat content of 60 kcal ad libitum for 28 weeks (high-fat diet). Rats were randomly assigned to undergo Roux-en Y gastric bypass (RYGB) or sham operation. Eight OLETF rats underwent sham operation (SHAM), and twelve OLETF rats underwent RYGB surgery.

The RYGB procedure was as follows. A rat was anesthetized using 2% isoflurane gas and the abdomen was shaved. Iodine was applied to the midline of the abdomen, and the skin and peritoneum were opened. The esophagogastric junction was identified, cut, and sutured. The jejunum approximately 34 cm away from the duodenum, was measured, cut, and anastomosed to the esophagus. Thereafter, the blind end of the gastric limb was anastomosed approximately 18 cm away from the anastomosed esophagojejunostomy site. Three limbs were formed, including the esophagojejunostomy limb (approximately 18 cm), the gastric limb (approximately 34 cm), and the common limb (approximately 24 cm). In the sham operation (SHAM), the peritoneal membrane was opened, and the jejunum was cut and then anastomosed.

12 weeks after RYGB surgery, the livers of the sham operation (SHAM) group and the RYGB surgery group were harvested and sampled, and then subjected to H&E staining and Masson's trichrome staining (a staining method for fibrosis assessment). As a result, it was found that the progression of steatohepatitis and fibrosis was suppressed in the RYGB surgery group compared to the sham operation group.

### Example 2: Identification of Factors Affecting Steatohepatitis Alleviation Caused by Increased Glucose Metabolism in Intestine After Gastrectomy in Type 2 Diabetes Rat Animal Model OLETF Rat

12 weeks after the rat underwent surgery in the same manner as in Example 1, 1 mCi of fluorodeoxyglucose (FDG) was injected into the tail vein of the rat. 1 hour later, the rat was sacrificed and the intestines were incised. For the RYGB rat, limbs (esophageal limb, gastric limb, and common branch) of the intestinal lumen were irrigated with normal saline, and FDG uptake in each intestinal segment was assessed by autoradiography.

The haustrum and intestines were aligned on the upper side of grid paper, and autoradiography images were acquired using a BAS 1040 imaging plate. Through autoradiography, intestinal tissues with the highest FDG uptake and the lowest FDG uptake were selected from the esophageal limb. In the sham operation group, jejunum tissue was selected.

The tissue segment with the highest FDG uptake, the tissue segment with the lowest FDG uptake, and the jejunum tissue segment in the sham operation group were cut, and RNA was extracted therefrom and then subjected to high-throughput RNA sequencing (Macrogen, Korea).

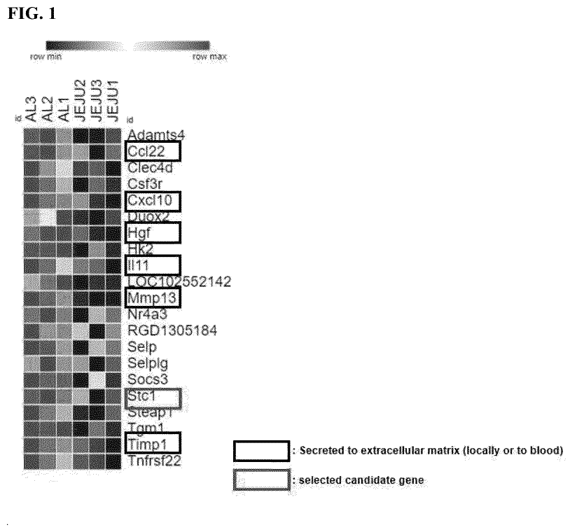

As a result of RNA sequencing, among the top 20 genes with the greatest difference in expression among transcripts, genes that translate proteins that can be secreted locally or to the blood were selected. Thereamong, it was found that expression of the STC-1 (stanniocalcin-1) gene significantly increased in the tissue with high FDG uptake in the RYGB surgery group compared to the tissues with low FDG uptake in the sham operation group and the RYGB surgery group (FIG. 1).

Based on these results, plasma was obtained from blood collected from the inferior vena cava and hepatic portal vein of each rat, and the amount of STC-1 protein was measured by performing ELISA analysis.

The analysis was performed using an ELISA kit (Cat #ab213829, Abcam, MA, USA) according to the manufacturer's instructions.

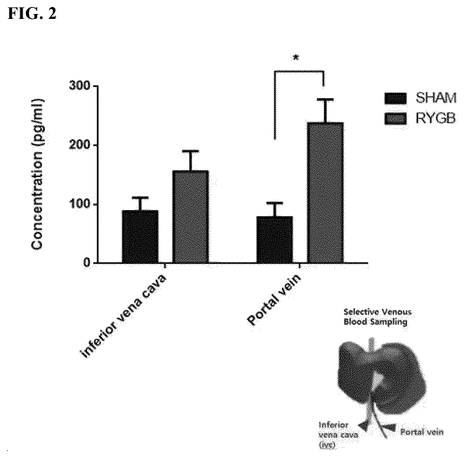

As a result, as shown in FIG. 2, it was found that the amount of STC-1 increased in both the inferior vena cava and the hepatic portal vein in the RYGB surgery group compared to the sham operation (SHAM) group. In particular, it was found that the concentration of STC-1 was significantly high in the hepatic portal vein (blood passage from the intestines to the liver).

In addition, RNA was extracted from the alimentary limb (AL) segments with and without increased FDG uptake (as assessed by autoradiography) and from the liver tissue, and the expression of STC-1 was measured by qRT-PCR. As primers for measuring the expression of STC-1, the primers of SEQ ID NOS: 1 and 2 were used (Table 1).

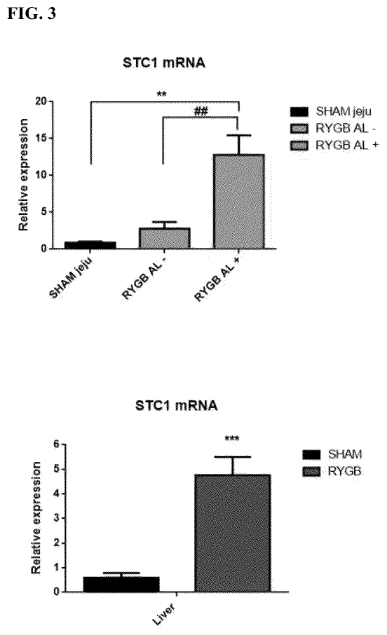

As a result, as shown in FIG. 3, it was found that STC-1 expression increased in RYGB AL− (− indicates the segment without increased FDG uptake) and AL+ (+ indicates the segment with increased FDG uptake) compared to the sham operation group (SHAM). In addition, it was found that the mRNA expression of STC-1 increased in the liver of the rat that underwent RYGB surgery compared to the sham operation group (SHAM).

### Example 3: Evaluation of Fat Metabolism, Immune Response, and Degree of Fibrosis in Liver Cell Lines by Treatment with Recombinant Human STC-1 Protein

For culture of human hepatocyte HepG2 cells (the Seoul National University Hospital Cell Line Bank, Seoul, Republic of Korea) and human hepatic stellate LX-2 cells (Merck, USA), the cells were placed in Dulbecco's modified Eagle medium (DMEM) supplemented with 10% fetal bovine serum (FBS) and penicillin-streptomycin, and maintained at 37° C. in a humidified atmosphere containing 10% CO₂ and 90% air. The cells were subcultured every 3 to 4 days.

The cells were seeded into 6-well dishes at a density of 2×10⁵ cells/dish and cultured. When the cell density reached 1.5×10⁵ cells/cm², the cells were plated for 6 days, and then the culture was used. For a free fatty acid-treated group, palmitic acid and oleic acid (Sigma) at a ratio of 2:1 were conjugated with 5% free fatty acid-free BSA, and then the cells were treated therewith. For a free fatty acid+STC-1 treated group, the cells were treated with free fatty acid and 100 ng/ml of STC-1 (Cat #RD172095100, Biovendor R&D). After 18 hours, RNA was extracted from the cells, and changes in expression of fat metabolism-related genes and immune response (inflammation)-related genes were measured by qRT-PCR.

Total RNA was extracted from the HepG2 cells using the RNeasy Mini kit (Qiagen) and synthesized into cDNA using a ReverTra Ace (Toyobo, Osaka, Japan). For PCR, the forward and reverse primers shown in Table 1 below were used for amplification.

TABLE 1

| Primer name | Sequence | SEQ ID NO. |
|---|---|---|
| rSTC1_F | CACGAGGCGGAACAGAATGA | 2 |
| rSTC1_R | CCAACCTGTAGGGCACTGTT | 3 |
| hSTC1_F | ATGGCGACCACCAAAGTCAA | 4 |
| hSTC1_R | GCAGTGACGCTCATAAGGGA | 5 |
| r18S_F | TGTCAATCCTGTCCGTGTCC | 6 |
| r18S_R | ACGGACCAGAGCGAAAGCAT | 7 |
| hET1_F | AGAGTGTGTCTACTTCTGCCA | 8 |
| hET1_R | CTTCCAAGTCCATACGGAACAA | 9 |
| hIL33_F | AATCAGGTGACGGTGTTG | 10 |
| hIL33_R | ACACTCCAGGATCAGTCTTG | 11 |
| hFN1_F | CGGTGGCTGTCAGTCAAAG | 12 |
| hFN1_R | AAACCTCGGCTTCCTCCATAA | 13 |
| hCOL4_F | TGGACAGGATGGATTGCCAG | 14 |
| hCOL4_R | GGGGACCTCTTTCACCCTTAAAA | 15 |
| heNOS_F | TGATGGCGAAGCGAGTGAAG | 16 |
| heNOS_R | ACTCATCCATACACAGGACCC | 17 |
| hIL11_F | CGAGCGGACCTACTGTCCTA | 18 |
| hIL_R | GCCCAGTCAAGTGTCAGGTG | 19 |
| hLECT2_F | GCTGGTCTGATTTCTACCGCA | 20 |
| hLECT2_R | TCCAGCAGAGCACAAGATGTC | 21 |
| hTIE1_F | AAGCAGACAGACGTGATCTGG | 22 |
| hTIE1_R | GCACGATGAGCCGAAAGAAG | 23 |
| hITE2_F | GGACTTCCTTCGCAAGAGCC | 24 |
| hTIE2_R | CGAAGTGAAGGAGCTGCTGG | 25 |
| mET1_F | GAGCGCGTCGTACCGTATG | 26 |
| mET1_R | ACTGACATCTAACTGCCTGGT | 27 |
| mFN1_F | ATGTGGACCCCTCCTGATAGT | 28 |
| mFN1_R | GCCCAGTGATTTCAGCAAAGG | 29 |
| mIL33_F | ATTTCCCCGGCAAAGTTCAG | 30 |
| mIL33_R | AACGGAGTCTCATGCAGTAGA | 31 |
| mCOL4_F | GAAAAAGGCGAACAAGGTCTTC | 32 |
| mCOL4_R | CAGGAGGCCCTCTATCACCA | 33 |
| mIL11_F | GCGCTGTTCTCCTAACCCG | 34 |
| mIL11_R | GAGTCCAGACTGTGATCTCCG | 35 |
| meNOS_F | TCAGCCATCACAGTGTTCCC | 36 |
| meNOS_R | ATAGCCCGCATAGCGTATCAG | 37 |
| mLECT2_F | GGACGTGTGACAGCTATGGC | 38 |
| mLECT2_R | TCCCAGTGAATGGTGCATACA | 39 |
| mTIE1_F | AATGCCGCGTATCGACTTCT | 40 |
| mTIE1_R | GACCACAAGCTGACGGCTCT | 41 |
| mTIE2_F | GAGGCGATCCCTGCAAACAA | 42 |
| mTIE2_R | GTCCAGGCCCTGAGTTCTTC | 43 |
| mActa2_F | ATGCTAACAACGTCCTGTCA | 44 |
| mActa2_R | AGAGTACTTGCGTTCTGGAG | 45 |
| mCcl2_F | CCCAATGAGTAGGCTGGAGA | 46 |
| mCcl2_R | TCTGGACCCATTCCTTCTTG | 47 |
| mChop_F | GAAGCCTGGTATGAGGATCT | 48 |
| mChop_R | ACTGACCACTCTGTTTCCGT | 49 |
| mClec4F_F | GACCATTGGATTGGGCTCAC | 50 |
| mClec4F_R | CTCGCTCTCCGTTCCTATGT | 51 |
| mCol1a1_F | GCTCCTCTTAGGGGCCACT | 52 |
| mCol1a1_R | CCACGTCTCACCATTGGGG | 53 |
| mIL6_F | CAACCACGGCCTTCCCTACT | 54 |
| mIL6_R | TTCTGCAAGTGCATCATCGTTGT | 55 |
| mRPL32_F | CAGTCAGACCGATATGTGAA | 56 |
| mRPL32_R | TAGAGGACACATTGTGAGCA | 57 |
| mTNF_F | GCTACGACGTGGGCTACAG | 58 |
| mTNF_R | CCCTCACACTCAGATCATCTTCT | 59 |
| mTGFb1_F | TGAGTGGCTGTCTTTTGACG | 60 |
| mTGFb1_R | GGTTCATGTCATGGATGGTG | 61 |
| mTiMP1_F | CTTGGTTCCCTGGCGTACTC | 62 |
| mTIMP1_R | ACCTGATCCGTCCACAAACAG | 63 |
| mOpn_F | AGGACAACAACGGAAAGGGC | 64 |
| mOpn_R | ATCCGACTGATCGGCACTCT | 65 |
| mPpara_F | GGATGTCACACAATGCAATTCG | 66 |
| mPpara_R | TCACAGAACGGCTTCCTCAGGT | 67 |
| mPparg_F | ATCCCTGGTTTCATTAACCT | 68 |
| mPparg_R | GCTCCATAAAGTCACCAAAG | 69 |
| mIL1b_F | GCAACTGTTCCTGAACTCAACT | 70 |
| mIL1b_R | ATCTTTTGGGGTCCGTCAACT | 71 |
| mIL10_F | GCTCTTACTGACTGGCATGAG | 72 |
| mIL10_R | CGCAGCTCTAGGAGCATGTG | 73 |
| mLox_F | TCTTCTGCTGCGTGACAACC | 74 |
| mLox_R | GAGAAACCAGCTTGGAACCAG | 75 |
| mCol4a1_F | CTGGCACAAAAGGGACGAG | 76 |
| mCol4a1_R | ACGTGGCCGAGAATTTCACC | 77 |
| mCol4a2_F | GACCGAGTGCGGTTCAAAG | 78 |
| mCol4a2_R | CGCAGGGCACATCCAACTT | 79 |
| mCol1a2_F | GTAACTTCGTGCCTAGCAACA | 80 |
| mCol1a2_R | CCTTTGTCAGAATACTGAGCAGC | 81 |
| hACLY_F | TCGGCCAAGGCAATTTCAGAG | 82 |
| hACLY_R | CGAGCATACTTGAACCGATTCT | 83 |
| hACACA_F | ATGTCTGGCTTGCACCTAGTA | 84 |
| hACACA_R | CCCCAAAGCGAGTAACAAATTCT | 85 |
| hFASN_F | AAGGACCTGTCTAGGTTTGATGC | 86 |
| hFASN_R | TGGCTTCATAGGTGACTTCCA | 87 |
| hME1_F | GAGTGCTGACATCTGACATTGA | 88 |
| hME1_R | TTGGCTTCCGAAACACCAAAC | 89 |
| hELOVL3_F | CTGTTCCAGCCCTATAACTTCG | 90 |
| hELOVL3_R | GAATGAGGTTGCCCAATACTCC | 91 |
| hELOVL6_F | AACGAGCAAAGTTTGAACTGAGG | 92 |
| hELOVL6_R | TCGAAGAGCACCGAATATACTGA | 93 |
| hSCD_F | TCTAGCTCCTATACCACCACCA | 94 |
| hSCD_R | TCGTCTCCAACTTATCTCCTCC | 95 |
| hMOGAT1_F | AAAGTGTGTCCTACATGGTAAGC | 96 |
| hMOGAT1_R | TGATCCTTCAGGGTTGTCAGTT | 97 |
| hMOGAT2_F | ACACTTGCTGTCCTACAGTTTG | 98 |
| hMOGAT2_R | GAGGAGCCAGAATCTTGTAAACA | 99 |
| hDGAT1_F | TATTGCGGCCAATGTCTTTGC | 100 |
| hDGAT1_R | CACTGGAGTGATAGACTCAACCA | 101 |
| hDGAT2_F | AGCAGGTGATCTTCGAGGAG | 102 |
| hDGAT2_R | CATGGGGCGAAACCAATGTA | 103 |
| hCIDEA_F | TTATGGGATCACAGACTAAGCGA | 104 |
| hCIDEA_R | TGCTCCTGTCATGGTTGGAGA | 105 |
| hCIDEC_F | CAGCAGCTCCTCGATGCTAC | 106 |
| hCIDEC_R | TCAGACAGGTCGGGATAAGGG | 107 |
| hCD36_F | GGCTGTGACCGGAACTGTG | 108 |
| hCD36_R | AGGTCTCCAACTGGCATTAGAA | 109 |
| hFABP1_F | AAGACAGTGGTTCAGTTGGAAG | 110 |
| hFABP1_R | TGAGTTCGGTCACAGACTTGAT | 111 |
| hSLC27a1_F | CTTCGATGGCTATGTCAGCGA | 112 |
| hSLC27a1_R | AGCACGTCACCTGAGAGGTAG | 113 |
| hPPARG_F | ACCAAAGTGCAATCAAAGTGGA | 114 |
| hPPARG_R | ATGAGGGAGTTGGAAGGCTCT | 115 |
| hLDLR_F | TCTGCAACATGGCTAGAGACT | 116 |
| hLDLR_R | TCCAAGCATTCGTTGGTCCC | 117 |
| hCPT1a_F | TCCAGTTGGCTTATCGTGGTG | 118 |
| hCPT1a_R | TCCAGAGTCCGATTGATTTTTGC | 119 |
| hCPT1b_F | CATGTATCGCCGTAAACTGGAC | 120 |
| hCPT1b_R | TGGTAGGAGCACATAGGCACT | 121 |
| hACADM_F | TGGATAACCAACGGAGGAAAAG | 122 |
| hACADM_R | CTGGGGTATCTGCTTCCACA | 123 |
| hACOX1_F | ACTCGCAGCCAGCGTTATG | 124 |
| hACOX1_R | AGGGTCAGCGATGCCAAAC | 125 |

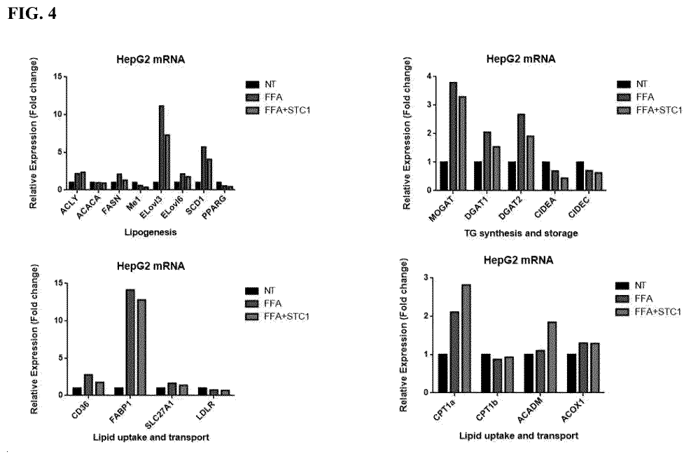

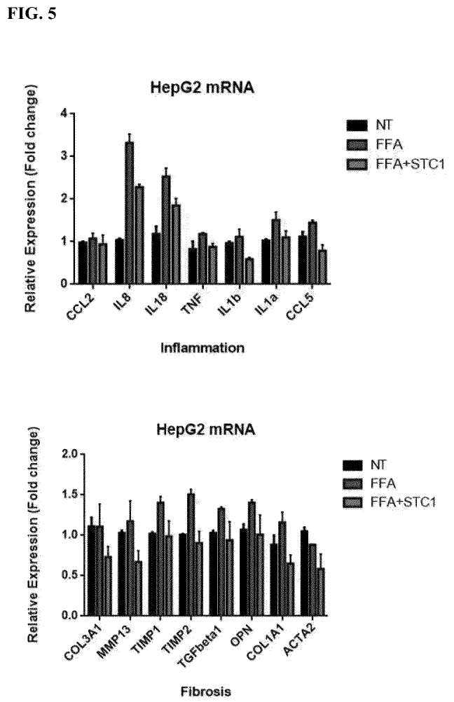

As a result, as shown in FIGS. 4 and 5, it was found that, when the HepG2 cells were treated with palmitic acid and oleic acid at a ratio of 2:1, the expression of fat metabolism-related genes increased, whereas, when the cells were treated with palmitic acid and oleic acid plus STC-1, the expression of fat metabolism-related genes (FIG. 4) and inflammation- and fibrosis-related genes (FIG. 5) decreased.

In addition, human hepatic stellate LX-2 cells (Merck, USA) that cause liver fibrosis were stimulated with TGF-beta to induce fibrosis and activation. First, the cells were treated with serum-free medium for 24 hours, and then incubated with 100 ng/ml of STC-1 or GDF-15 (Cat #957-GD R&D Systems, USA) 1 hour before treatment with TGF-beta. Next, the cells were stimulated by treatment with TGF-beta. After 18 hours of incubation, RNA was extracted and subjected to qRT-PCR using primers related to inflammation and fibrosis (Table 1).

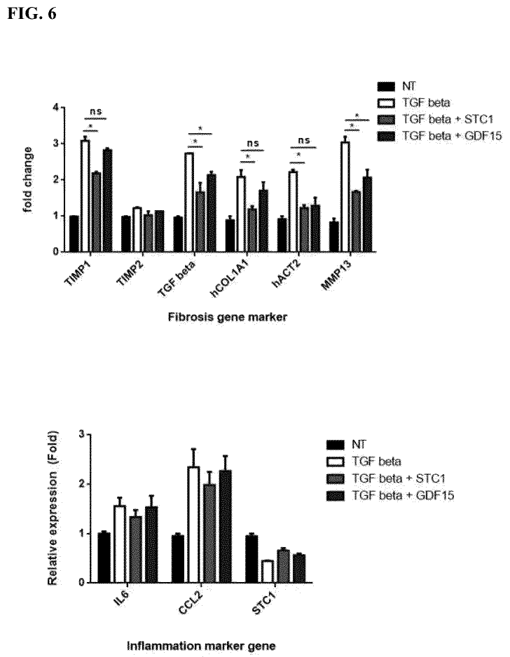

As a result, as shown in FIG. 6, it was found that inflammation and fibrosis marker genes significantly decreased in the STC-1 or GDF15 treated group compared to the TGF-beta treated group. In particular, it was found that the STC-1 treated group exhibited a better effect than the GDF15 treated group.

### Example 4: Examination of AMPKα Phosphorylation in Liver Cell Line Overexpressing STC-1STC-1

A cell line was generated by overexpressing STC-1 in human hepatocyte HepG2 cells (Seoul National University Hospital Cell Line Bank, Seoul, Republic of Korea).

HepG2 cells were seeded into 6-well dishes at a density of 2×10⁵ cells/dish and cultured. After 24 hours, the cells were transfected with a pCMV-STC-1 vector using PolyJet (Signagen, USA) to generate an STC-1-overexpressing cell line.

The pCMV-STC-1 vector was constructed by inserting an STC-1-encoding gene fragment (SEQ ID NO: 142) into a pCMV vector (Korea Human Gene Bank (KHGB), Republic of Korea).

After treating the generated STC-overexpressing cell line with palmitic acid, total protein was extracted from the cells and subjected to Western blot analysis using t-AMPKα antibody, p-AMPKα antibody and STC-1 as primary antibodies.

Total cell protein lysates were prepared and subjected to Western blot analysis according to standard procedures. Briefly, the cells were cooled on ice, washed twice with ice-cold phosphate-buffered saline, and lysed in buffer containing 1 mM phenylmethylsulfonyl fluoride and 1× protease inhibitors (Sigma-Aldrich). Protein concentration was measured using the BCA protein assay kit (Thermo Scientific).

An equal amount of protein from the cell lysate was separated by SDS-polyacrylamide gel electrophoresis and incubated with primary antibodies at 4° C. overnight. The blot was washed three times with TBST (Tris-buffered saline containing 0.05% Tween 20) and then incubated with horseradish peroxidase (HRP)-conjugated secondary antibodies at 25° C. for 1 hour. As the secondary antibodies, donkey anti-rabbit IgG-HRP antibody (1:2,000, Santa Cruz) and donkey anti-mouse IgG-HRP antibody (1:2,000, Santa Cruz) were used. Immunoreactivity was detected with a SuperSignal West Pico Chemiluminescent Substrate (Thermo Fisher Scientific, MA, USA).

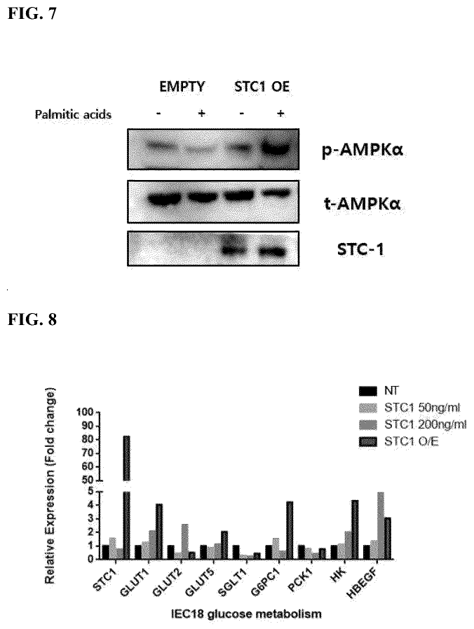

As a result, as shown in FIG. 7, it was found that, when the cells overexpressing STC-1 were treated with palmitic acid, phosphor AMPKα increased. When P-AMPKα increases, NASH is reduced through mitochondrial uncoupling action.

### Example 5: Evaluation of Effect of STC-1 Overexpression on Glucose Metabolism in Intestinal Epithelial Cell Line

Cells overexpressing STC-1 were generated by transforming intestinal epithelial IEC18 cells (Seoul National University Hospital Cell Line Bank, Seoul, Republic of Korea) with a pCMV-STC-1 vector. After 48 hours of culture, RNA was extracted from the cells. Meanwhile, wild-type IEC18 cells were treated with 50 ng/ml and 200 ng/ml of STC-1, and after 18 hours, RNA was extracted from the cells. The extracted RNA was subjected to qRT-PCR using primers capable of amplifying glucose metabolism-related genes.

TABLE 2

| Primer name | Sequence | SEQ ID NO. |
|---|---|---|
| rGLUT1_F | CTCATAGCTGGACGGCTGTT | 126 |
| rGLUT1_R | GGCCCAAGGTAGCTGGAAAT | 127 |
| rGLUT2_F | TTCAGCAACTGGGTCTGCAA | 128 |
| rGLUT2_R | AAGAACACGTAAGGCCCGAG | 129 |
| rGLUT5_F | GTGGTAGAGCTTTGGGGACG | 130 |
| rGLUT5_R | ACTATGCAAGCTGTGAGGCA | 131 |
| rSGLT1_F | AGTGGGCTGTACCAACATCG | 132 |
| rSGLT1_R | CAGGCCTCGAAGTCCGTTAG | 133 |
| rG6PC1_F | CCCAGACTAGAGATCCTGACAGAAT | 134 |
| rG6PC1_R | GCACAACGCTCTTTTCTTTTACC | 135 |
| rPCK1_F | GCCTGTGGGAAAACCAACCT | 136 |
| rPCK1_R | CACCCACACATTCAACTTTCCA | 137 |
| rHK_F | AGGGACGGATGTGTGTCAAC | 138 |
| rHK_R | CATCAAACACGGTCCGCAAG | 139 |
| rHB-EGF_F | GGAGAGTGCAGATACCTGAAGGA | 140 |
| rHB-EGF_R | GTCAGCCCATGACACCTCTGT | 141 |

As a result, as shown in FIG. 8, it was found that the expression of GLUT1 gene and hexokinase (HK) gene increased depending on the concentration of STC-1. In addition, it was found that the expression of GLUT1 gene and hexokinase (HK) gene was also increased by overexpression (O/E) of STC-1.

### Example 6: Evaluation of Effect of STC-1 Protein Administration in Mouse Model of Liver Fibrosis

While wild-type BL6 mice (OrientBio, Republic of Korea) were fed an MCD (methionine choline-deficient) diet for 8 weeks, recombinant human STC-1 protein or physiological saline (vehicle) was administered to the mice daily by intraperitoneal injection. Body weight was measured once a week, and AST and ALT levels in the serum from the mice were measured using an ELISA kit (SEO KWANG LABOTECH, Republic of Korea). Additionally, the level of Lect2 in the liver was measured by qPCR.

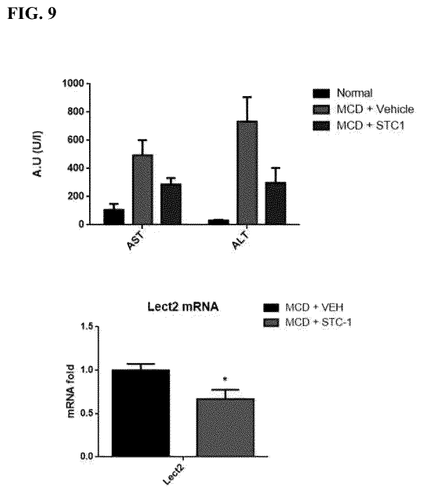

As a result, as shown in FIG. 9, it was found that the AST and ALT levels tended to decrease in the STC-1-administered group compared to the control (vehicle) group. A recent paper revealed that, even in cirrhosis patients, there are groups with normal ALT levels, and thus cirrhosis cannot be accurately distinguished based on ALT levels alone, and cirrhosis lesions can be more clearly distinguished by Lect2 levels (Xu, M et al., Cell, 178 (6), 1478-1492, 2019).

In this Example, as shown in FIG. 9, it was found that not only the ALT level but also the Lect2 mRNA level significantly decreased in the STC-1-administered group, indicating that fibrosis was alleviated by STC-1 administration.

In addition, after 8 weeks, the livers of the mice were harvested, and then RNA was extracted therefrom and subjected to qPCR to measure the expression level of fibrosis- and inflammation-related genes.

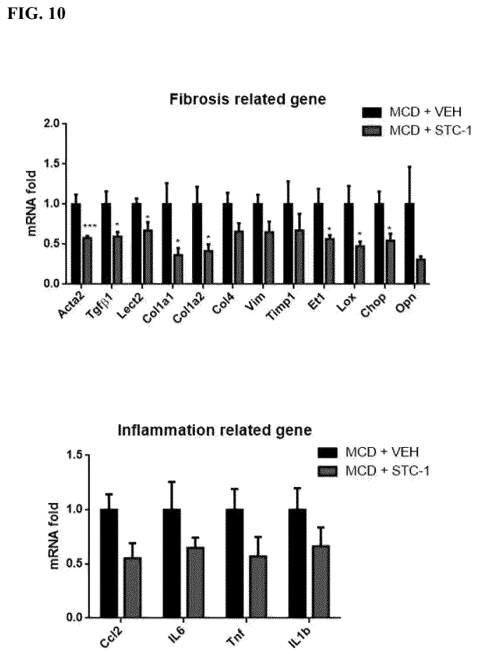

As a result, as shown in FIG. 10, it was found that the expression of fibrosis- and inflammation-related genes decreased in the MCD diet-fed mouse group to which STC-1 was administered.

In addition, the degree of fibrosis was measured by Sirius red staining. The harvested mouse liver tissue was fixed with formaldehyde and made into a paraffin block which was then sectioned into 5 MM thick slices and transferred onto slides. The sections were subjected to Sirius Red staining according to the manufacturer's instructions and imaged under a microscope, and the Sirius red-positive area was measured by image J quantification.

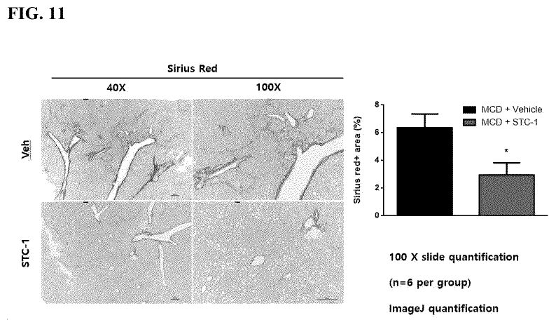

As a result, as shown in FIG. 11, it was found that the Sirius red positive area significantly decreased in the MCD diet-fed mouse group to which STC-1 was administered.

### Example 7: Evaluation of Anti-Inflammatory Effect of hSTC-1-Overexpressing Hepatocytes

Human hepatocyte HepG2 cells (Seoul National University Hospital Cell Line Bank, Seoul, Republic of Korea) were seeded into the upper well of a Transwell insert, and mouse macrophage Raw264.7 cells (monocyte cells, ATCC TIB-71) were seeded into the lower well. The HepG2 cells in the upper well were transfected with a pCMV-STC-1 vector to overexpress STC-1, and the Raw264.7 cells in the lower well were treated with LPS (100 ng/ml). After the cells were incubated for 48 hours, changes in expression of inflammatory cytokines were measured by qRT-PCR according to the method of Example 3.

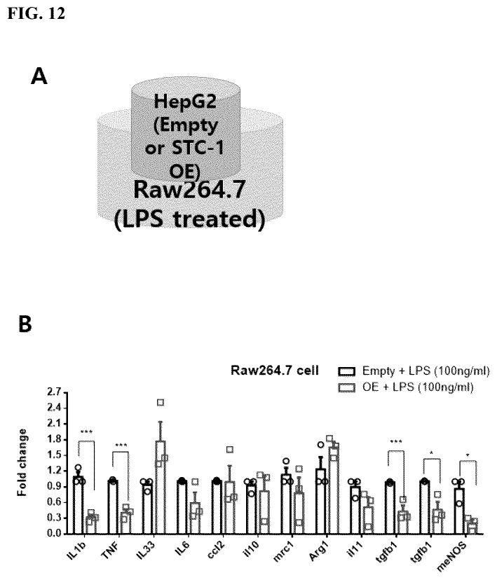

As a result, as shown in FIG. 12, it was found that inflammation induced by LPS was reduced by hSTC-1 secreted from the HepG2 cells overexpressing hSTC-1.

### Example 8: Examination of Changes in Inflammatory Cytokine Expression by hSTC-1 Treatment in Inflammation-Induced Macrophages

Mouse macrophage Raw264.7 cells were treated with LPS (100 ng/mL), hSTC-1 (100 ng/ml), and ATP (2 mM) for 6 hours to induce NRLP3 activation. Then, changes in expression of inflammatory cytokines IL-1beta, TNF, IL-6, Cc12 and TGF-beta were measured by qRT-PCR according to the method of Example 3.

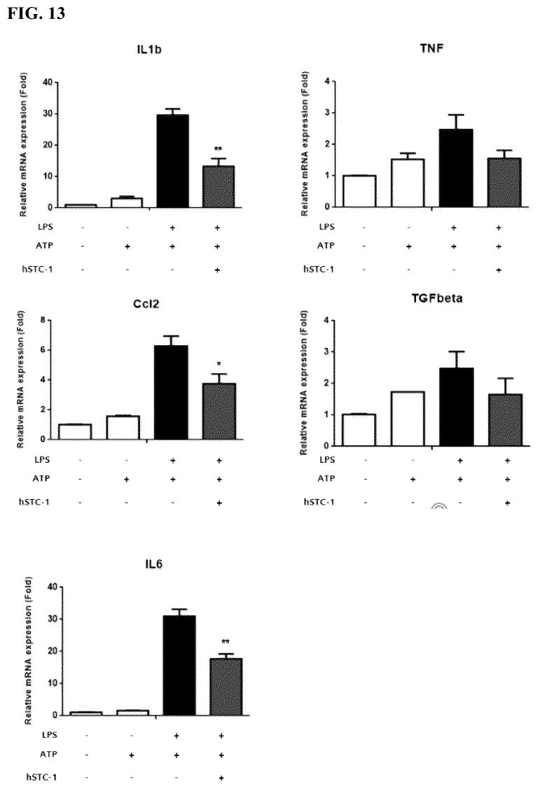

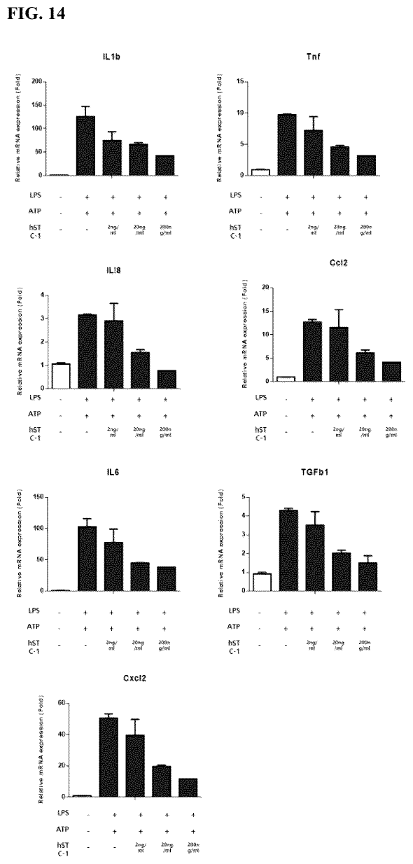

As a result, as shown in FIG. 13, it was found that inflammation induced by LPS was reduced by hSTC-1 treatment.

### Example 9: Evaluation of Anti-Inflammatory Effect After Treatment of Inflammation-Induced Macrophages with Different Concentrations of hSTC-1

Inflammation was induced by treating mouse macrophage Raw264.7 cells with LPS (100 ng/ml) and 2 ng/ml, 20 ng/ml and 200 ng/ml of hSTC-1 and treating the cells with ATP (2 mM) for 6 hours to promote inflammatory response. Then, changes in mRNA expression of inflammatory cytokines were measured by qRT-PCR according to the method of Example 3.

As a result, as shown in FIG. 14, it was found that inflammation was alleviated dependently on the concentration (dose) of hSTC-1 used for treatment.

### INDUSTRIAL APPLICABILITY

The STC-1 protein according to the present invention reduces the expression of liver fibrosis markers ALT and Lect2 and reduces the area of fibrotic tissue, in animal models of liver fibrosis, indicating that it has excellent effects on the prevention and treatment of liver fibrosis and, furthermore, cirrhosis.

Although the present invention has been described in detail with reference to specific features, it will be apparent to those skilled in the art that this description is only of a preferred embodiment thereof, and does not limit the scope of the present invention. Thus, the substantial scope of the present invention will be defined by the appended claims and equivalents thereto.

### Sequence Listing Free Text

Electronic file attached

1. A method of preventing or treating liver disease in a subject in need thereof, comprising administering to said subject a composition comprising STC-1 or a derivative thereof as an active ingredient.

2. The method according to claim 1, wherein the liver disease is selected from the group consisting of liver fibrosis, cirrhosis, acute hepatitis, chronic hepatitis, fatty liver, and liver cancer.

3. The method according to claim 1, wherein STC-1 consists of the amino acid sequence represented by SEQ ID NO: 1.

4. A health functional food for preventing or ameliorating liver disease comprising STC-1 or a derivative thereof as an active ingredient.

5. A health functional food for improving liver function comprising STC-1 or a derivative thereof as an active ingredient.
